(19) United States
(12) Patent Application Publication
(10) Pub. No.: US 2013/0266657 A1
(43) Pub. Date: Oct. 10, 2013

(54) MALODOR CONTROL COMPOSITIONS

(71) Applicant: Anita Trajkovska, Christiansburg, VA (US)

(72) Inventor: Anita Trajkovska, Christiansburg, VA (US)

(73) Assignee: NESTEC SA, Vevey (CH)

(21) Appl. No.: 13/881,530

(22) PCT Filed: Oct. 21, 2011
(86) PCT No.: PCT/US2011/001796

§ 371 Date: Jun. 18, 2013

Related U.S. Application Data
(60) Provisional application No. 61/455,837, filed on Oct. 27, 2010.

(51) Int. Cl. A61L 9/014; A01K 1/01

(52) U.S. Cl. CPC A61L 9/014; A01K 1/0107
USPC 424/490; 424/76.6; 424/489; 119/165

(57) ABSTRACT

The invention provides a malodor control composition including a porous material having (1) a porous structure having a surface area of from about 50 to about 1500 m2/g, (2) a pore diameter of from about 1 to about 200 nm, (3) a pore volume of from about 0.3 to about 1.5 cm3/g, and (4) a pronounced hydrophobicity with a surface contact angle greater than 100 degrees. The malodor control composition can be used as an animal litter or in conjunction with other animal litters to provide more effective control for removing odors resulting from animal waste.

### CROSS REFERENCE TO RELATED APPLICATIONS

This application claims priority to U.S. Provisional Application Ser. No. 61/455,837 filed Oct. 27, 2010, the disclosure of which is incorporated herein by this reference.

### BACKGROUND OF THE INVENTION

1. Field of the Invention

The invention relates generally to malodor control and particularly to malodor control compositions and animal litters including such malodor control compositions.

2. Description of Related Art

Malodor management is an issue in many fields, e.g., garbage and other wastes, animal housing, and animal litters. Effective control of malodors during litter box maintenance is one of the key consumer drivers associated with acceptance or non-acceptance of a particular litter. Most current litter box odor control strategies rely on strong fragrance applications prior to litter packaging and/or spraying deodorants around the litter box after use by an animal. These approaches mask the malodors, but can be viewed as too strong or offensive by some consumers. There is also some concern that the fragrance can be perceived negatively by animals such as cats, thereby causing cats to avoid the litter box.

To date, a variety of malodor counteractant (“MOC”) technologies have been proposed and used in different industries dealing with malodors (e.g., pig farms, cow farms, and poultry farms, waste landfields, etc.). Besides the masking mechanism via fragrances, different MOC technologies can be classified on the basis of their mechanism of action as described below.

MOCs acting via biological mechanisms. As it is known, malodor is usually generated by partial oxidation of the organic waste by bacteria. If the oxidation is complete (final products CO₂ and H₂O), there will be no malodor. Biological MOCs usually involve enzymes and bacteria. Bacterial strains produce specific enzymes that attack specific chemical units in the organic waste. In general, this mechanism is a slow-acting.

MOCs acting via physical mechanisms. There are two sub-classes in this category: (1) MOCs acting via adsorption—this is a physical adherence of the malodor molecules onto the MOC molecules as a result of the physical Van der Waals forces and (2) MOCs acting via physi-sorption or absorption—this is the physical penetration of the malodor substance into the inner molecular structures of the MOC product.

MOCs acting via anesthetization. These MOCs desensitize the olfactory senses so that no odor, good or bad, will be perceived. This mechanism is also known as “deodorizing by odor fatigue.” MOCs acting via chemical mechanism: If a malodor can be made to react chemically with the MOC, it would become a chemically-different compound that will smell differently. There are several sub-classes in this category, such as: (1) MOCs acting via bonding (chemi-sorption)—chemi-sorption involves the exchange or sharing of electrons between the malodor atoms and those of the MOC product. Sometimes, this affects the vapor pressure of the malodorants and (2) MOCs acting via oxidation-reduction—oxido-reduction using oxidizing agents, such as chlorine, sodium and calcium hypochlorite, chlorine dioxide, potassium permanganate and hydrogen peroxide. Ozone emitting devices are also used to oxidize the airborne malodor molecules.

MOCs acting via counteraction. A phenomenon that occurs when the proper two odors are physically in the same area, with the overall odor being reduced instead of increased. This method is termed neutralization, when no odor results, or reodorization, when a milder pleasant odor replaces the malodor. It works through Zwaardemaker pairs (conjugates), pairs of odorants that neutralize each other's respective odors.

As stated before, most of the commercially available litters use the fragrance for masking the malodors originated from pet waste. The fragrance is simply applied (e.g., by spraying or as a physical blend) before litter packaging, or as breakable fragrance scented balls (US20060185608A1). Other MOCs have been patented for odor control in litter applications. For example, US20050005869, U.S. Pat. No. 5,860,391, WO1990009099A1 and US20070017453 are related to use of activated carbon for odor control in animal litter, while U.S. Pat. No. 6,895,896 is related to the use of silica gel particles for odor control. Other compounds for odor control in litter applications include baking soda (U.S. Pat. No. 6,955,136), sodium fluoride (U.S. Pat. No. 5,097,799), guanidine salts (U.S. Pat. No. 4,957,063), halo sulfonamide compounds (US2006/0075975A1), oxidizing agent (U.S. Pat. No. 5,005,520) and boron-containing compounds (U.S. Pat. No. 5,094,190). Nevertheless, there is a continuing need for new compositions that effectively control malodors.

### SUMMARY OF THE INVENTION

It is, therefore, an object of the invention to provide malodor control compositions.

It is another object of the invention to provide malodor control compositions useful for controlling malodors caused by wastes such as animal waste.

It is a further object of the invention to provide methods for controlling malodors.

It is another object of the invention to provide animal litters incorporating malodor control compositions.

It is a further object of the invention to provide kits useful for producing animal litters useful for controlling malodors.

These and other objects are achieved using malodor control compositions that include one or more porous materials having (1) a porous structure having a surface area of from about 50 to about 1500 m²/g, (2) a pore diameter of from about 1 to about 200 nm, (3) a pore volume of from about 0.3 to about 1.5 cm³/g, and (4) a pronounced hydrophobicity with a surface contact angle greater than 100 degrees. In addition, the use of these specified porous materials in litter formulations enables effective control of malodors originating from animal waste, particularly in a litter box. Particularly, the pores of certain size and their chemical affinity act as effective traps for the compounds that cause malodors. Such animal litter formulations incorporating the specified porous materials do not require application of strong fragrances for masking malodors or other malodor counteracting technology.

Additional and further objects, features, and advantages of the invention will be readily apparent to those skilled in the art.

### BRIEF DESCRIPTION OF THE DRAWINGS

FIG. 1 shows a malodor control composition in an embodiment of the invention.

FIG. 2 shows an animal litter in an embodiment of the invention.

### DETAILED DESCRIPTION OF THE INVENTION

### Definitions

The term “animal litter(s)” means a composition that is suitable for use as an animal litter (e.g., managing animal waste) but that can also be used for any other suitable purpose. For example, an animal litter of the invention could be used to absorb a chemical spill, absorb an oil spill, create traction on a slippery surface, and the like.

The term “malodor(s)” means any malodor but particularly a malodor from animal waste such as feces and urine.

All percentages expressed herein are by weight of the total weight of the composition unless expressed otherwise.

As used throughout, ranges are used herein in shorthand, so as to avoid having to set out at length and describe each and every value within the range. Any appropriate value within the range can be selected, where appropriate, as the upper value, lower value, or the terminus of the range.

As used herein and in the appended claims, the singular form of a word includes the plural, and vice versa, unless the context clearly dictates otherwise. Thus, the references “a”, “an”, and “the” are generally inclusive of the plurals of the respective terms. For example, reference to “a porous material” or “a method” includes a plurality of such “porous materials” or “methods”. Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context. Where used herein the term “examples,” particularly when followed by a listing of terms is merely exemplary and illustrative, and should not be deemed to be exclusive or comprehensive.

The compositions, products, methods and other advances disclosed here are not limited to particular methodology, protocols, and reagents described herein because, as the skilled artisan will appreciate, they may vary. Further, the terminology used herein is for the purpose of describing particular embodiments only, and is not intended to, and does not, limit the scope of that which is disclosed or claimed.

### The Invention

In one aspect, the invention provides malodor control compositions. The compositions comprise a porous material comprising (1) a porous structure having a surface area of from about 50 to about 1500 m2/g, (2) a pore diameter of from about 1 to about 200 nm, (3) a pore volume of from about 0.3 to about 1.5 cm3/g, and (4) a pronounced hydrophobicity with a surface contact angle greater than 100 degrees. The invention is useful for controlling malodors from wastes such as animal wastes and for controlling malodors in facilities characterized by malodors, e.g., in litter boxes, animal bedding, animal housing, storage facilities, bathrooms, gymnasiums, and the like.

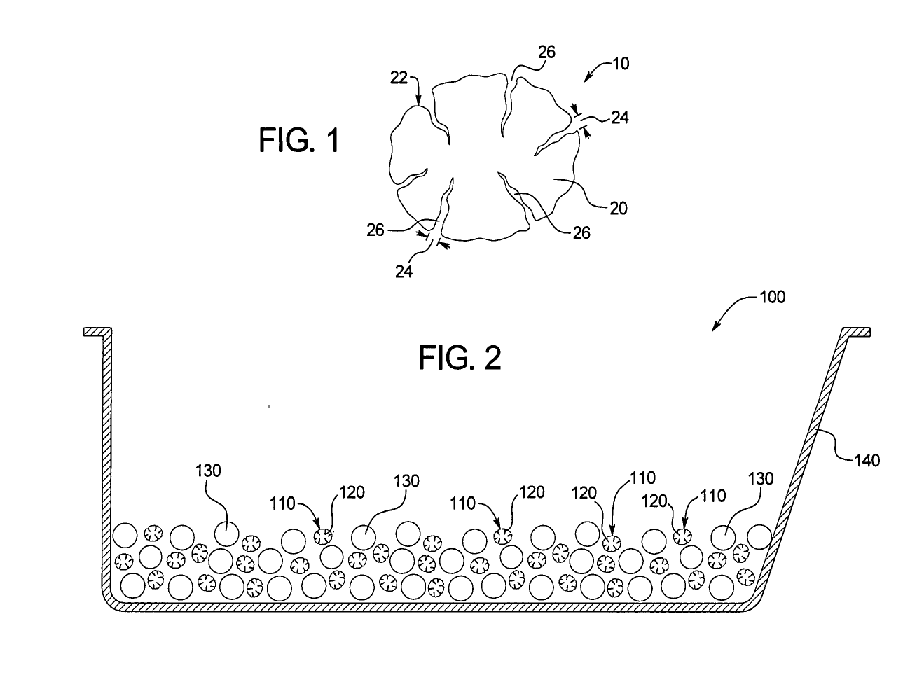

As shown in FIG. 1, the invention provides a malodor control composition 10. The malodor control composition 10 includes one or more porous materials 20 having (1) a porous structure 22 having a surface area of from about 50 to about 1500 m²/g, (2) a pore diameter 24 of from about 1 to about 200 nm, (3) a pore volume 26 of from about 0.3 to about 1.5 cm³/g, and (4) a pronounced hydrophobicity with a surface contact angle greater than 100 degrees. Malodor control composition 10 can be applied directly to or placed in close proximity to feces, urine, bedding, housing, and other malodor sources. Close proximity is any distance that permits the composition to control the malodors. In general, close proximity is within 1 to about 200 centimeters (cm), preferably within about 5 to about 100 cm, more preferably within about 10 to about 80 cm. In various embodiments, when the malodor is all or part of a gas, the gas is passed through the malodor composition to remove the malodors.

Malodor control composition 10 can be made of any color, shape, and size material having these specific characteristics to allow for incorporation in or addition to a variety of litters or substrates. For example, malodor control composition 10 can be in the shape of particulates having any suitable particle sizes to meet the specified criteria. Alternatively, malodor control composition 10 can be in the shape of spheres, cylinders, cubes, cones, and the like having varying surface characteristics, including surfaces that are smooth, rough, concave, convex, and the like.

The invention is based upon the discovery that the porous materials having the previously described characteristics are especially suited to interact with compounds or compositions that cause malodors via adsorption and/or absorption mechanisms. Animal litters containing the porous materials having the tailored pores can be advantageously used for controlling malodors originating from pet waste.

Generally, as previously discussed, the adsorption mechanism can be defined as the attachment of an analyte (adsorbate) at the surface of a solid material (adsorbent), which can occur at the external and internal surfaces of the adsorbent. The pore diameter is also important, e.g., it has to be sufficiently large that the molecules to be adsorbed can migrate through the pore to the adsorbing surface. In addition, the pore size distribution is an important factor that determines the effectiveness of an adsorbent. The adsorption itself can be a physical (or physisorption) and a chemical (or chemisorptions). Physisorption involves interactions, such as Van der Waals forces and/or electrostatic forces (permanent dipole-permanent dipole, charge-charge, or charge-dipole). Chemisorption is obtained when the analyte reacts with the adsorbent surface, i.e., adsorbed molecules undergo some chemical interaction with the adsorbent.

Porous materials that differed in their physical properties, such as pore volume, pore size, pore size distribution, pore character (e.g., open pores, closed pores, inter-connected/throughput pores), and specific surface area, were tested for their potential to adsorb and/or absorb the malodors in a litter box. Because the adsorption capability of a particular porous material is affected by the chemical structure as well, the tested porous materials were selected to cover a wide range of chemical structures, and thus, variety of chemical affinities and surface energies. As a general principle in the area of physical adsorption, hydrophobic or non-polar materials are attracted more to hydrophobic surfaces and hydrophilic or polar materials are attracted more to hydrophilic surfaces. Further testing details are discussed the Examples section below.

In an embodiment, malodor control composition 10 can also include other malodor control materials such as, e.g., perfumes, sorbent materials, hydrophobic nanozeolites (U.S. Pat. No. 6,660,713), silica gel (U.S. Pat. No. 6,860,234; U.S. Pat. No. 6,578,521; and U.S. Pat. No. 6,543,385), and the like, partially or fully integrated with malodor control composition 10. The sorbent material can be activated carbon, baking soda, or a combination thereof. In an embodiment, the sorbent material can be from about 0.5% to about 99.5% by weight of the malodor control composition. In another embodiment, the sorbent material can be from about 1% to about 10% by weight of the malodor control composition. In yet another embodiment, the sorbent material can be from about 2% to about 8% by weight of the malodor control composition.

In another aspect shown in FIG. 2, the invention provides an animal litter 100. The animal litter includes one or more animal litters 130 and one or more porous materials 110. Porous material 110 has (1) a porous structure 120 having a surface area of from about 50 to about 1500 m²/g, (2) a pore diameter of from about 1 to about 200 nm, (3) a pore volume of from about 0.3 to about 1.5 cm³/g, and (4) a pronounced hydrophobicity with a surface contact angle greater than 100 degrees (e.g., as shown in FIG. 1).

As further shown in FIG. 2, animal litter 100 can be contained in a device 140 suitable for containing animal litter and suitable for use by an animal when excreting animal waste. Suitable devices 140 include an animal litter box. Device 140 can be, for example, the litter boxes disclosed in US20090250014, US20090272327, US20090000560, US20070277740, and U.S. Pat. No. 7,628,118. Device 140 can include any suitable amount of animal litter 100 as desired by the user.

Animal litter 130 can be any suitable material that functions as an animal litter. In an embodiment, animal litter 130 can be one or more clays, woods, papers, grains, corncobs, seeds, or combinations thereof. In another embodiment, animal litter 130 can be swelling clay, non-swelling clay, silica gel or a combination thereof (e.g., U.S. Pat. No. 6,860,234, U.S. Pat. No. 6,543,385, and U.S. Pat. No. 6,578,521). In a preferred embodiment, the animal litter is a mixture of one or more malodor compositions of the invention and a clumping litter, e.g., the clumping litter disclosed in U.S. Pat. No. 6,887,570.

Porous material 110 can be combined with such other animal litter 130 in any suitable amount to produce the mixture of animal litter 100. For example, porous material 110 can include from about 5 to about 95% of the mixture, preferably from about 10 to about 90%. In one embodiment, the mixture includes about 50% of porous material 110 and about 50% of silica gel, clumping animal litter, non-clumping animal litter, or combination thereof. In another embodiment, the mixture includes about 90% of porous material 110 and about 10% silica gel. In a further embodiment, the mixture includes about 70% of porous material 110 and about 30% clumping litter.

In another embodiment, animal litter 130 and/or porous material 110 includes activated carbon. The activated carbon can range from about 0.01% to about 6% by weight of animal litter 130 and/or porous material 110.

In yet another embodiment, animal litter 130 and/or porous material 110 includes baking soda. The baking soda can range from about 0.01% to about 6% by weight of animal litter 130 and/or porous material 110.

In still another embodiment, animal litter 130 and/or porous material 110 further includes a partial or complete coating of one or more swelling clays. For example, the swelling clay can be bentonite. The swelling clay can range from about 5 to about 40% by weight of animal litter 130 and/or porous material 110.

In an alternative aspect, the invention provides a method for making an animal litter that can be used to control malodor. The method comprises combining one or more animal litters with one or more porous materials including (1) a porous structure having a surface area of from about 50 to about 1500 m²/g, (2) a pore diameter of from about 1 to about 200 nm, (3) a pore volume of from about 0.3 to about 1.5 cm³/g, and (4) a pronounced hydrophobicity with a surface contact angle greater than 100 degrees.

In various embodiments the malodor control compositions and litters may contain other ingredients such as compounds or compositions that add functionality to the malodor control compositions or litters. For example, the malodor control compositions may contain or be admixed with ingredients selected from the group consisting of surfactants, perfumes, preservatives, anti-microbials, de-foaming agents, antifoaming agents, bacteriocides, fungicides, antistatic agents, insect and moth repellents, colorants, bluing agents, antioxidants and mixtures thereof.

In yet another aspect, the invention provides a kit suitable for containing malodor control compositions useful for managing animal waste. The kit comprises in separate containers in a single package or in separate containers in a virtual package, as appropriate for the kit component, the malodor control composition as described in any of the embodiments herein and one or more of (1) a device suitable for containing the malodor control composition and suitable for use by an animal when excreting animal waste (e.g., a litter box), (2) a device suitable for handling animal waste that has been deposited with the malodor control composition (e.g., a scoop for removing animal feces from a litter (e.g., U.S. Pat. No. 7,523,973) or a rake suitable for arranging an animal litter in a litter box or other container), (3) a different animal litter (e.g., a different animal litter suitable for creating a mixture of the malodor control composition and such different animal litter), (4) a fragrance, (5) a sorbent material, (6) instructions for how to use the malodor control composition to manage animal waste, (7) instructions for how to use the malodor control composition to control odor in a variety of situations, and (8) instructions for how to dispose of the malodor control composition (e.g., how to dispose of the litter in an environmentally friendly manner, particularly after it has been used).

In still another aspect, the invention provides a kit suitable for containing animal litters useful for managing animal waste. The kit comprises in separate containers in a single package or in separate containers in a virtual package, as appropriate for the kit component, the animal litter as described in any of the embodiments herein and one or more of (1) a device suitable for containing the animal litter and suitable for use by an animal when excreting animal waste (e.g., a litter box), (2) a device suitable for handling animal waste that has been deposited with the animal litter (e.g., a scoop for removing animal feces from a litter (e.g., U.S. Pat. No. 7,523,973) or a rake suitable for arranging an animal litter in a litter box or other container), (3) a different animal litter (e.g., a different animal litter suitable for creating a mixture of the animal litter and such different animal litter), (4) a fragrance, (5) a sorbent material, (6) instructions for how to use the animal litter to manage animal waste, (7) instructions for how to use the animal litter to control odor in a variety of situations, and (8) instructions for how to dispose of the animal litter (e.g., how to dispose of the litter in an environmentally friendly manner, particularly after it has been used).

When the kit comprises a virtual package, the kit can be limited to instructions in a virtual environment in combination with one or more physical kit components. The kits may contain the kit components in any of various combinations and/or mixtures. For example, in one embodiment, the kit contains a package containing the malodor control composition and a scoop suitable for removing animal waste from the malodor control composition. In one embodiment, the kit contains a package containing the animal litter and a scoop suitable for removing animal waste from the animal litter.

In an aspect, the invention provides a means for communicating information about or instructions for using the malodor control composition as described in any of the embodiments herein for one or more of (1) managing animal waste such as animal urine and feces, (2) controlling odor, (3) controlling moisture, (4) controlling microorganisms, and (5) controlling dust, the means including a document, digital storage media, optical storage media, audio presentation, or visual display containing the information or instructions.

In another aspect, the invention provides a means for communicating information about or instructions for using the animal litter as described in any of the embodiments herein for one or more of (1) managing animal waste such as animal urine and feces, (2) controlling odor, (3) controlling moisture, (4) controlling microorganisms, and (5) controlling dust, the means including a document, digital storage media, optical storage media, audio presentation, or visual display containing the information or instructions.

In certain embodiments, the communication means is a displayed website, a visual display kiosk, a brochure, a product label, a package insert, an advertisement, a handout, a public announcement, an audiotape, a videotape, a DVD, a CD-ROM, a computer readable chip, a computer readable card, a computer readable disk, a USB device, a FireWire device, a computer memory, and any combination thereof.

Useful information includes one or more of (1) methods and techniques for training or adapting an animal to use the malodor control composition/animal litter, (2) functional or other properties of the malodor control composition/animal litter, and (3) contact information for to use by a consumer or others if there is a question about the malodor control composition/animal litter and their uses. Useful instructions include methods for cleaning and disposing of the malodor control composition/animal litter. The communication means is useful for instructing on the benefits of using the malodor control composition/animal litter and communicating the approved methods for using the malodor control composition/animal litter for an animal.

In another aspect, the invention provides packages useful for containing malodor control compositions or animal litters of the invention. In one embodiment, the package comprises at least one material suitable for containing malodor control compositions of the invention and a label affixed to the material containing a word or words, picture, design, acronym, slogan, phrase, or other device, or combination thereof, that indicates that the package contains such malodor control compositions, e.g., information about the malodor control composition's density and/or its physical, functional, and related properties. Typically, such device comprises the words “controls odors” or “malodor control composition” or an equivalent expression printed on the material. In preferred embodiments, the package further comprises one or more malodor control compositions of the invention. In another embodiment, the package comprises at least one material suitable for containing animal litters of the invention and a label affixed to the material containing a word or words, picture, design, acronym, slogan, phrase, or other device, or combination thereof, that indicates that the package contains such animal litters.

Typically, such device includes the words “animal litter for malodor control” or “reduces odors” or an equivalent expression printed on the package. In preferred embodiments, the package further comprises one or more animal litters of the invention, e.g., information about the animal litter's density and/or its physical, functional, and related properties).

Any package configuration and packaging material suitable for containing malodor control compositions and animal litters are useful in the invention, e.g., a bag, box, bottle, can, pouch, and the like manufactured from paper, plastic, foil, metal, and the like. In various embodiments, the package further comprises at least one window that permit the package contents to be viewed without opening the package. In some embodiments, the window is a transparent portion of the packaging material. In others, the window is a missing portion of the packaging material.

In an aspect, the invention provides methods for managing malodorous waste. The methods comprise contacting a malodorous waste with one or more malodor control compositions described in any of the embodiments herein. In a preferred embodiment, the malodorous waste is an animal waste, preferably a waste from a dog or cat. In some embodiments, the method further comprises disposing the malodor control composition.

In another aspect, the invention provides methods for managing animal waste. The methods comprise contacting the animal waste with an animal litter as described in any of the embodiments herein. In some embodiments, the methods further comprise disposing the animal litter. Generally, the malodor control composition/animal litter is placed in a litter box or other suitable container and the animal is allowed to deposit its waste (urine or feces) so that it comes in contact with the malodor control composition/animal litter. If desirable, the malodor control composition/animal litter can be placed on contact with the waste after the waste is deposited, e.g., on a lawn.

In other aspects, the invention provides methods for controlling malodors. The methods comprise exposing a malodorous material to at least one malodor control composition of the invention.

In other aspects, the invention provides methods for controlling malodors from animal waste. The methods comprise exposing the animal waste to at least one malodor control composition of the invention.

In other aspects, the invention provides methods for controlling malodors from animal waste. The methods comprise exposing the animal waste with at least one animal litter of the invention. In these aspects, exposing the materials or wastes means contacting the materials or wastes with the malodor control compositions or litters or placing the materials or wastes in close proximity to the malodor control compositions or litters.

Malodorous materials and wastes include any material that has an undesirable odor. Examples include animal wastes from porcine, bovine, equine, ovine, canine, feline, and other animals. Malodorous materials also include garbage, landfill wastes, malodorous gases, and the like.

### EXAMPLES

The invention can be further illustrated by the following examples, although it will be understood that the examples are included merely for purposes of illustration and are not intended to limit the scope of the invention unless otherwise specifically indicated.

### Example 1

Litter formulation A1 was prepared by mixing 142.5 grams conventional clay litter A with the following characteristics: density=40-47 lbft³, particle size distribution=4-60 mesh, and random shaped particles and 7.5 grams base-treated porous cross-linked polystyrene beads (5 wt % beads) with the following characteristics: specific surface area=800-1000 m²/g, pore volume=1-1.1 ml/g, specific gravity=1.04 g/ml, spherical beads=16-50 mesh, pore size d₅₀ (meso-pores and macro-pores)=85-95 nm and d₅₀ (micro-pores)=1.5 nm, and strong-base functionality (Hypersol-Macronet resin MN-400, Purolite Company).

Twenty (20) grams cat fecal matter and 20 ml cat urine were added to 150 grams of litter formulation A (the fecal matter was immersed/cover with the clay particles). After two days, the fecal matter was taken out from the litter formulation Al and the soiled litter formulation A was stirred well. 3 grams of such soiled litter formulation were placed in a vial for GC-MS evaluation of the soiled litter headspace. Sampling of the litter headspace was done with SPME fiber (PADS/CAR/DAB) at temperature of 80° C. for 30 min. After desorption of the extracted compounds from the SPME fiber at 250° C., the GC run using non-polar column (HP-1 ms) was performed and MS detector was used for headspace compounds' identification.

Control sample A was prepared with 150 grams of conventional clay litter A, treated with fecal matter and urine in the same way as described above for litter formulation A1, and evaluated via GC-MS headspace analysis. The results from GC-MS analytical evaluation of the soiled litter headspace of control sample A and soiled litter formulation A1 are given in Table 1 and Table 2, respectively. Only the chromatographic peaks with a good match with the peak library (peak quality 80%) are given in the Tables.

TABLE 1
Headspace Composition Of The Control Sample A (Conventional Clay Litter
A) Assessed With GC-MS Analytical Tool Coupled SPME Fiber Sampling

| RT | Library/ID | Qual | Area |
|---|---|---|---|
| 1.5714 | Carbon disulfide | 90 | 2831382 |
| 1.8112 | Propanal, 2-methyl- (CAS) | 80 | 3731964 |
| 2.6933 | Acetic acid | 90 | 2606545 |
| 3.0701 | 3-Buten-1-ol, 3-methyl- | 80 | 3573584 |
| 3.3741 | Toluene | 81 | 3553421 |
| 3.6439 | 2-Butenal, 3-methyl- | 90 | 15823336 |
| 3.8537 | Hexanal (CAS) | 96 | 5191435 |
| 4.6801 | Cyclotrisiloxane, hexamethyl- (CAS) | 90 | 21906297 |
| 5.2325 | Benzene, 1,3-dimethyl- (CAS) | 93 | 4575378 |
| 7.0138 | Benzaldehyde (CAS) | 95 | 66424771 |
| 7.1123 | Oxime-, methoxy-phenyl- | 83 | 57696669 |
| 7.7289 | Nonane, 2-methyl- (CAS) | 90 | 7358185 |
| 8.08 | Benzene, 1,2,3-trimethyl- | 91 | 23544690 |
| 8.2941 | Octanal | 80 | 5561442 |
| 8.5895 | Decane (CAS) | 94 | 9363505 |
| 8.8636 | Benzene, methyl(1-methylethyl)- (CAS) | 86 | 30819461 |
| 9.0906 | Cyclohexene, 1-methyl-4-(1-methylethenyl)-, (R)- (CAS) | 90 | 13050104 |
| 9.1847 | Nonane, 2,6-dimethyl- | 90 | 11221425 |
| 10.0283 | Decane, 5-methyl- | 94 | 8148713 |
| 10.2595 | Phenol, 4-methyl- | 94 | 68102020 |
| 11.0603 | Undecane (CAS) | 96 | 13878103 |
| 11.7539 | 11H-Dibenzo[b,e][1,4]diazepin-11-one, 5-(3- | 90 | 17211378 |
| | aminopropyl)-5,10-dihydro- | | |
| 12.0708 | Acetic acid, phenylmethyl ester | 97 | 8306225 |
| 12.4176 | Azulene (CAS) | 81 | 5382815 |
| 12.6146 | Nonadecane | 87 | 8336819 |
| 12.8116 | Cyclopentasiloxane, decamethyl- | 91 | 18229189 |
| 13.1927 | Cyclododecane | 95 | 5524675 |
| 13.4496 | Dodecane (CAS) | 97 | 8768386 |
| 13.6509 | Heptadecane | 90 | 6518802 |
| 13.7408 | Undecane, 2,4-dimethyl- | 97 | 5338649 |
| 13.8179 | Undecane, 2,6-dimethyl- | 87 | 13347986 |
| 13.9977 | Undecane, 4,8-dimethyl- | 93 | 10677630 |
| 14.67 | Tetradecane (CAS) | 80 | 7640167 |
| 14.7898 | Dodecane, 4,6-dimethyl- | 87 | 16527785 |
| 15.0382 | 5-[4-(Dimethylamino)benzylideneamino]-2-phenyl-2H- | 83 | 11766457 |
| | benzotriazole | | |
| 15.2181 | Isobornyl acetate | 97 | 17071655 |
| 15.5734 | Pentadecane | 94 | 12180804 |
| 15.7105 | Pentadecane | 81 | 11740106 |
| 15.8775 | Pentadecane | 94 | 7851004 |
| 16.2372 | Eicosane | 86 | 5776170 |
| 18.0185 | 1H-3a,7-Methanoazulene, 2,3,4,7,8,8a-hexahydro-3,6,8,8- | 95 | 7890904 |
| | tetramethyl-, [3R-(3.alpha.,3a.beta.,7.beta.,8a.alpha.)]- | | |
| | (CAS) | | |
| 19.2303 | Hexadecane, 2,6,10,14-tetramethyl- (CAS) | 89 | 6971639 |
| 19.9668 | Tetracosane (CAS) | 90 | 13761064 |
| 20.836 | Octadecane (CAS) | 93 | 4385956 |

RT = retention time; Library/ID = compound name; Qual = GC peak quality (match with library); Area = GC peak area

TABLE 2
Headspace Composition Of Litter Formulation A1 (5 Wt
% Base-Treated MN-400 Beads With Specific Surface Area =
800-1000 M2/G, Pore Size D50 (Meso- And Macro-Pores) =
85-95 Nm And D50 (Micro-Pores) = 1.5 Nm Added
To Clay Litter A) Assessed With GC-MS Analytical Tool
Coupled SPME Fiber Sampling

| RT | Library/ID | Qual | Area |
|---|---|---|---|
| 2.0382 | Ethane, 1,2-dichloro- (CAS) | 91 | 10545330 |
| 4.6802 | Cyclotrisiloxane, hexamethyl- (CAS) | 94 | 18183722 |
| 7.0695 | Oxime-, methoxy-phenyl- | 90 | 55482777 |
| 8.8593 | Cyclotetrasiloxane, octamethyl- | 91 | 12187522 |
| 12.8073 | Cyclopentasiloxane, decamethyl- | 91 | 5623228 |

Referring to the Tables 1 and 2, the data shows that most of the compounds, odorous and non-odorous compounds, that are present in the soiled litter headspace of the control sample A (Table 1) are absorbed/adsorbed by the porous beads present in the litter formulation A1 (Table 2). Even the air headspace sampling (extraction) temperature of 80° C. (which means heating the litter formulation at 80° C.) did not result in releasing (desorption) of the “trapped” compounds from the pores of used beads. Therefore, porous beads with proper pore sizes, pore size distribution and surface activity can successfully control the malodors in a litter box.

### Example 2

Litter formulation B1 was prepared by mixing 161.5 grams of conventional clay litter B (significantly different in chemical composition from the conventional clay litter A used in Example 1) with the following characteristics: density=46-52 lb/ft³, particle size distribution=4-60 mesh, and random shaped particles, and 8.5 grams base-treated porous cross-linked polystyrene beads (5 wt % beads) with the following characteristics: specific surface area=800-1000 m²/g, pore volume=1-1.1 mug, specific gravity=1.04 g/ml, spherical beads=16-50 mesh, pore size d₅₀ (meso- and macro-pores)=85-95 nm and d₅₀ (micro-pores)=1.5 nm (Hypersol-Macronet resin MN-400, Purolite Company).

Litter formulation B1 was treated with cat fecal matter and urine in the same way as litter formulation A1, described in Example 1. GC-MS headspace analysis of soiled litter formulation B1 was performed using the procedure described in Example 1.

Control sample B was prepared with 170 grams conventional clay litter B, treated with cat waste and analyzed via GC-MS as described in Example 1. The results from the GC-MS analysis of the headspace composition of control sample B and litter formulation B1 are given in Table 3 and Table 4, respectively.

TABLE 3
Headspace Composition Of Control Sample B (Conventional
Clay Litter B) Assessed With GC-MS Analytical
Tool Coupled SPME Fiber Sampling

| RT | Library/ID | Qual | Area |
|---|---|---|---|
| 1.5758 | Carbon disulfide | 90 | 3281824 |
| 3.8581 | Hexanal (CAS) | 90 | 3768621 |
| 4.6802 | Cyclotrisiloxane, hexamethyl- (CAS) | 90 | 20229922 |
| 5.1041 | Butanoic acid (CAS) | 87 | 12534398 |
| 7.0139 | Benzaldehyde (CAS) | 97 | 31529685 |
| 7.1038 | Oxime-, methoxy-phenyl- | 83 | 32361143 |
| 7.8403 | Glycine, | 28 | 5517871 |
| | N-[4-[(trimethylsilyl)oxy]benzoyl]-, | | |
| | methyl ester | | |
| 8.0715 | Phenol (CAS) | 87 | 22832651 |
| 8.2899 | Octanal (CAS) | 91 | 4536141 |
| 8.8637 | Cyclotetrasiloxane, octamethyl- (CAS) | 91 | 17294388 |
| 9.0864 | Benzenemethanol (CAS) | 93 | 11121859 |
| 9.4503 | Benzene, 1,2-diethyl- (CAS) | 95 | 7268641 |
| 9.5959 | Benzene, 1,4-diethyl- (CAS) | 97 | 3090280 |
| 10.0841 | Decane, 3,7-dimethyl- | 81 | 9911201 |
| 10.2725 | Phenol, 4-methyl- (CAS) | 94 | 87116990 |
| 10.7906 | Nonanal | 93 | 10700374 |
| 11.7498 | Sulfamoxole | 90 | 12639154 |
| 12.6104 | Undecane, 2-methyl- | 83 | 5350593 |
| 12.8117 | Cyclopentasiloxane, decamethyl- | 91 | 23458022 |
| 13.1928 | Cyclododecane | 95 | 6844685 |
| 13.4497 | Dodecane (CAS) | 95 | 6812326 |
| 13.7409 | Undecane, 2,4-dimethyl- | 93 | 3475632 |
| | $$ 2,4-Dimethylundecane | | |
| 13.818 | Undecane, 2,6-dimethyl- | 94 | 8583512 |
| | $$ 2,6-Dimethylundecane | | |
| | $$ 2,6-Dimethylundecene | | |
| 13.9935 | Undecane, 4,8-dimethyl- | 87 | 8151490 |
| 14.5373 | Octadecane (CAS) | 80 | 4509019 |
| 14.6701 | Nonadecane (CAS) | 81 | 5772821 |
| 14.8199 | 1H-Indole (CAS) | 94 | 42032090 |
| 15.3851 | Pentadecane | 86 | 39379394 |
| 15.5735 | Pentadecane | 91 | 10049263 |
| 15.7063 | Pentadecane | 90 | 11245425 |
| 15.8819 | Pentadecane | 80 | 12224816 |
| 16.2373 | Eicosane (CAS) | 83 | 5061275 |
| 16.3829 | Dodecane | 83 | 26141546 |
| 16.5884 | Pentadecane | 87 | 10842977 |
| 17.1322 | Tridecane, 2-methyl- | 87 | 4382370 |
| 17.6717 | 1-Tetradecene (CAS) | 97 | 6172446 |
| 17.8901 | Tetradecane (CAS) | 97 | 6145331 |
| 18.3697 | Tridecane, 3-methyl- | 93 | 4334848 |
| 19.2261 | Hexadecane (CAS) | 93 | 12826597 |
| 19.7742 | Tetradecane, 2,6,10-trimethyl- | 81 | 7185864 |
| 19.8256 | Nonadecane (CAS) | 89 | 4601021 |
| 19.8727 | Eicosane | 87 | 5792137 |
| 19.9754 | Eicosane (CAS) | 86 | 51293738 |
| 20.1553 | Tetradecane (CAS) | 90 | 9329327 |
| 20.2709 | Octadecane | 91 | 6171310 |
| 20.6605 | Octadecane | 90 | 7372644 |
| 20.8404 | Octadecane (CAS) | 81 | 25726846 |
| 21.0245 | Octadecane (CAS) | 93 | 8490504 |
| 21.1958 | Docosane (CAS) | 87 | 4782560 |
| 24.0176 | Eicosane | 93 | 13187854 |
| 24.7755 | Eicosane (CAS) | 89 | 19002589 |
| 27.9913 | Sulfur, mol. (S8) (CAS) | 94 | 79280885 |

TABLE 4
Headspace Composition Of Litter Formulation B1 (5 Wt
% Base-Treated MN-400 Beads With Specific Surface Area =
800-1000 M2/G, Pore Size D50 (Meso- And Macro-Pores) =
85-95 Nm And D50 (Micro-Pores) = 1.5 Nm Added
To Clay Litter B) Assessed With GC-MS Analytical Tool
Coupled SPME Fiber Sampling

| RT | Library/ID | Qual | Area |
|---|---|---|---|
| 2.034 | Ethane, 1,2-dichloro- | 95 | 4119492 |
| 4.6802 | Cyclotrisiloxane, hexamethyl- | 90 | 18465158 |
| 7.0739 | Oxime-, methoxy-phenyl- | 83 | 62523779 |
| 8.8594 | Cyclotetrasiloxane, octamethyl- (CAS) | 91 | 13433665 |
| 11.7412 | 11H-Dibenzo[b,e][1,4]diazepin-11-one, | 95 | 8647849 |
| | 5-(3-aminopropyl)-5,10-dihydro- | | |
| 12.8074 | Pentadecane, 2,6,10,14-tetramethyl- | 93 | 5814965 |
| | (CAS) | | |

Referring to the Tables 3 and 4, the data shows that most of the compounds, odorous and non-odorous compounds, that are present in the soiled litter headspace of the control sample B (Table 3) are absorbed/adsorbed by the porous beads present in the litter formulation B1 (Table 4). Even the air headspace sampling (extraction) temperature of 80° C. (which means heating the litter formulation at 80° C.) did not result in releasing (desorption) of the “trapped” compounds from the pores of used beads. Therefore, porous beads with proper pore sizes, pore size distribution and surface activity can successfully control the malodors in a litter box.

### Example 3

Litter formulation A2-1 was prepared by mixing 142.5 grams conventional clay litter A (described in Example 1) and 7.5 grams non-functionalized (hydrophobic) porous cross-linked polystyrene beads (5 wt % beads) with the following characteristics: specific surface area=800-1000 m²/g, pore volume=1-1.1 ml/g, specific gravity=1.04 g/ml, spherical beads=16-50 mesh, pore size d₅₀ (meso- and macro-pores)=85-95 nm and d₅₀ (micro-pores)=1.5 nm (Hypersol-Macronet resin MN-200, Purolite Company). Such formulation was treated with the cat fecal matter and urine and evaluated via GC-MS analysis, as described in Example 1. The results for headspace composition are given in Table 5.

TABLE 5
Headspace Composition Of Litter Formulation A2-1 (5
Wt % Non-Functionalized/Hydrophobic MN-200 Beads With
Specific Surface Area = 800-1000 M2/G, Pore Size
D50 (Meso- And Macro-Pores) = 85-95 Nm And D50
(Micro-Pores) = 1.5 Nm Added To Clay Litter A)
Assessed With GC-MS Analytical Tool Coupled SPME Fiber Sampling

| RT | Library/ID | Qual | Area |
|---|---|---|---|
| 1.8499 | Hexane | 81 | 4190688 |
| 2.034 | Ethane, 1,2-dichloro- | 96 | 1.01E+08 |
| 4.6803 | Cyclotrisiloxane, hexamethyl- | 90 | 22052092 |
| 7.0482 | Oxime-, methoxy-phenyl- | 83 | 56052768 |
| 8.8595 | Cyclotetrasiloxane, octamethyl- (CAS) | 91 | 15075651 |
| 11.7369 | Sulfamoxole | 87 | 5999713 |
| 12.8074 | Cyclopentasiloxane, decamethyl- | 91 | 6413262 |

Referring to the Table 5, the data shows that most of the compounds, odorous and non-odorous compounds, that are present in the soiled litter headspace of the control sample A (Table 1) are absorbed/adsorbed by the porous beads present in the litter formulation A2-1 (Table 5) where the porous beads were of similar physical characteristics but more hydrophobic than those in sample A2 (Table 2). Again showing that porous beads with proper pore sizes, pore size distribution and surface activity can successfully control the malodors in a litter box.

### Example 4

Litter formulation A3 was prepared by mixing 142.5 grams conventional clay litter A (described in Example 1) and 7.5 grams non-functionalized (hydrophobic) porous cross-linked polystyrene beads (5 wt % beads) with the following characteristics: specific surface area=800-1000 m²/g, pore volume=0.6-0.8 ml/g, specific gravity=1.04 g/ml, spherical beads=16-50 mesh, pore size d₅₀ (meso- and macro-pores)=30-40 nm and d₅₀ (micro-pores)=1.4 nm (Hypersol-Macronet resin MN-250, Purolite Company). Such formulation was treated with the cat fecal matter and urine and evaluated via GC-MS analysis, as described in Example 1, and the results for headspace composition are given in Table 6.

TABLE 6
Headspace Composition Of Litter Formulation A3 (5 Wt
% Non-Functionalized/Hydrophobic MN-250 Beads With Specific
Surface Area = 800-1000 M2/G, Pore Size D50 (Meso-
And Macro-Pores) = 30-40 Nm And D50 (Micro-Pores) =
1.4 Nm Added To Clay Litter A) Assessed With GC-MS
Analytical Tool Coupled SPME Fiber Sampling

| RT | Library/ID | Qual | Area |
|---|---|---|---|
| 1.8497 | Hexane | 83 | 4121319 |
| 4.68 | Cyclotrisiloxane, hexamethyl- (CAS) | 90 | 16428698 |
| 7.0052 | Oxime-, methoxy-phenyl- | 90 | 45889089 |
| 8.8592 | Cyclotetrasiloxane, octamethyl- | 91 | 11887300 |
| 11.7367 | 11H-Dibenzo[b,e][1,4]diazepin-11-one, | 95 | 3737032 |
| | 5-(3-aminopropyl)-5,10-dihydro- | | |
| 12.8072 | Cyclopentasiloxane, decamethyl- | 91 | 5493277 |
| 21.6581 | 2,2,4-Trimethyl-1,3-pentanediol | 86 | 3765913 |
| | diisobutyrate | | |

Referring to the Table 6, the data shows that most of the compounds, odorous and non-odorous compounds, that are present in the soiled litter headspace of the control sample A (Table 1) are absorbed/adsorbed by the porous beads present in the litter formulation A3 where the porous beads were of slightly lower porosity but more hydrophobic than those in sample A2 (Table 2). Therefore, porous beads with proper pore sizes, pore size distribution and surface activity can successfully control the malodors in a litter box.

### Example 5

Litter formulation A4 was prepared by mixing 142.5 grams conventional clay litter A (described in Example 1) and 7.5 grams moderately polar divinylbenzene N-vinylpyrolidine beads with the following characteristics: density=0.32 g/mL, specific surface area=525 m²/g, particle size distribution=80-100 mesh (Porapak R, Water Corp).

Such formulation was treated with the cat fecal matter and urine and evaluated via GC-MS analysis, as described in Example 1, and the results for headspace composition are given in Table 7.

TABLE 7
Headspace Composition Of Litter Formulation A4 (5 Wt %
Moderately Polar Porapak R Beads With Specific Surface
Area = 800-1000 M2/G Added To Clay Litter A) Assessed
With GC-MS Analytical Tool Coupled SPME Fiber Sampling

| RT | Library/ID | Qual | Area |
|---|---|---|---|
| 3.2844 | Pentane, 2 3,4-trimethyl- | 87 | 3424417 |
| 3.3743 | Toluene | 95 | 7098735 |
| 4.6803 | Cyclotrisiloxane, hexamethyl- | 90 | 14237694 |
| 7.3137 | Benzene, 1-ethyl-4-methyl- (CAS) | 93 | 5575007 |
| 7.3565 | Benzene, 1-ethyl-2-methyl- | 94 | 4501909 |
| 8.457 | Benzene, (2-methylpropyl)- | 87 | 8452377 |
| 8.5212 | Benzene, (1-methylpropyl)- (CAS) | 94 | 15741873 |
| 8.8681 | Cyclotetrasiloxane, octamethyl- (CAS) | 91 | 10744518 |
| 9.6046 | Benzene, 1,3-diethyl- | 97 | 1.45E+09 |
| 9.7459 | Benzene, 1,4-diethyl- (CAS) | 97 | 8.22E+08 |
| 9.8187 | Benzene, 1,2-diethyl- | 97 | 2.05E+08 |
| 9.93 | Benzene, 1,4-diethyl- | 97 | 12282998 |
| 10.2768 | Benzene, 1-ethenyl-3-ethyl-, mixt. | 93 | 4817840 |
| | with 1-ethenyl-4-ethylbenzene (CAS) | | |
| 11.737 | 11H-Dibenzo[b,e][1,4]diazepin-11-one, | 95 | 2708253 |
| | 5-(3-aminopropyl)-5,10-dihydro- | | |
| 11.9854 | Naphthalene, 1,2,3,4-tetrahydro- (CAS) | 93 | 5564070 |
| 12.7475 | Benzene, 1,3,5-trimethyl-2-propyl- | 81 | 8089985 |
| 12.8075 | Cyclopentasiloxane, decamethyl- | 90 | 5351416 |
| 12.9745 | 3-ETHYLACETOPHENONE | 95 | 7325420 |
| 13.6296 | Benzene, 1,3,5-triethyl- | 97 | 4081784 |
| 14.1135 | 4-Hydroxy-2-methylacetophenone | 86 | 10399696 |
| 14.3875 | Ethanone, 1-(4-ethylphenyl)- (CAS) | 97 | 1.06E+08 |
| 14.7986 | Ethanone, 1-(4-ethylphenyl)- (CAS) | 97 | 42194917 |

Referring to the Table 7, the data shows that the more polar beads of otherwise similar physical characteristics failed to absorb/adsorb as much of the malodor compounds found in the control sample A (Table 1) when compared to the more hydrophobic beads in Examples 1 to 4, 6 and 7. These beads will not control malodors in a litter box.

### Example 6

Litter formulation B2-1 was prepared by mixing 161.5 grams conventional clay litter B (described in Example 2) and 8.5 grams non-functionalized (hydrophobic) porous cross-linked polystyrene beads (5 wt % beads) with the following characteristics: specific surface area=800-1000 m²/g, pore volume=1-1.1 ml/g, specific gravity=1.04 g/ml, spherical beads=16-50 mesh, pore size d₅₀ (meso- and macro-pores)=85-95 nm and _{d50} (micro-pores)=1.5 nm (Hypersol-Macronet resin MN-200, Purolite Company). Such formulation was treated with the cat fecal matter and urine and evaluated via GC-MS analysis, as described in Example 1, and the results for headspace composition are given in Table 8.

TABLE 8
Headspace Composition Of Litter Formulation B2-1 (5
Wt % Non-Functionalized/Hydrophobic MN-200 Beads With
Specific Surface Area = 800-1000 M2/G, Pore Size
D50 (Meso- And Macro-Pores) = 85-95 Nm And D50
(Micro-Pores) = 1.5 Nm Added To Clay Litter B)
Assessed With GC-MS Analytical Tool Coupled SPME Fiber Sampling

| RT | Library/ID | Qual | Area |
|---|---|---|---|
| 2.0339 | Ethane, 1,2-dichloro- | 96 | 95389362 |
| 4.6759 | Cyclotrisiloxane, hexamethyl- | 90 | 18646962 |
| 7.031 | Oxime-, methoxy-phenyl- | 83 | 38453544 |
| 8.8594 | Cyclotetrasiloxane, octamethyl- (CAS) | 91 | 13801697 |
| 11.7369 | Pentadecane, 2,6,10,14-tetramethyl- (CAS) | 95 | 8246324 |
| 12.8074 | Cyclopentasiloxane, decamethyl- | 91 | 6745920 |

Referring to the Table 8, the data shows that that most of the compounds, odorous and non-odorous compounds, that are present in the soiled litter headspace of the control sample B (Table 3) are absorbed/adsorbed by the porous beads present in the litter formulation B2-1 (Table 8) where the porous beads were of similar physical characteristics but more hydrophobic than those in sample B2 (Table 4). Therefore, such beads can successfully control the malodors in a litter box.

### Example 7

Litter formulation B3 was prepared by mixing 161.5 grams conventional clay litter B (described in Example 2) and 8.5 grams non-functionalized (hydrophobic) porous cross-linked polystyrene beads (5 wt % beads) with the following characteristics: specific surface area=800-1000 m²/g, pore volume=0.6-0.8 ml/g, specific gravity=1.04 g/ml, spherical beads=16-50 mesh, pore size d₅₀ (meso- and macro-pores)=30-40 nm and d₅₀ (micro-pores)=1.4 nm (Hypersol-Macronet resin MN-250, Purolite Company). Such formulation was treated with the cat fecal matter and urine and evaluated via GC-MS analysis, as described in Example 1, and the results for headspace composition are given in Table 9.

TABLE 9
Headspace Composition Of Litter Formulation B3 (5 Wt
% Non-Functionalized/Hydrophobic MN-250 Beads With Specific
Surface Area = 800-1000 M2/G, Pore Size D50 (Meso-
And Macro-Pores) = 30-40 Nm And D50 (Micro-Pores) =
1.4 Nm Added To Clay Litter B) Assessed With GC-MS
Analytical Tool Coupled SPME Fiber Sampling

| RT | Library/ID | Qual | Area |
|---|---|---|---|
| 1.8498 | Hexane | 87 | 2383828 |
| 4.6759 | Cyclotrisiloxane, hexamethyl- | 90 | 16488022 |
| 7.0352 | Oxime-, methoxy-phenyl- | 83 | 37123555 |
| 8.8594 | Cyclotetrasiloxane, octamethyl- (CAS) | 91 | 11801686 |
| 11.7326 | Sulfamoxole | 91 | 7022296 |
| 12.8074 | Cyclopentasiloxane, decamethyl- | 91 | 6001018 |

Referring to the Table 9, the data shows that that most of the compounds, odorous and non-odorous compounds, that are present in the soiled litter headspace of the control sample B (Table 3) are absorbed/adsorbed by the porous beads present in the litter formulation B3 where the porous beads were of slightly lower porosity but more hydrophobic than those in sample B2 (Table 4). Therefore, such beads can successfully control the malodors in a litter box.

### Example 8

Litter formulation B4 was prepared by mixing 161.5 grams conventional clay litter B (described in Example 2) and 8.5 grams moderately polar divinylbenzene N-vinylpyrolidine beads with the following characteristics: density=0.32 g/mL, specific surface area=525 m²/g, and particle size distribution=80-100 mesh (Porapak R, Water Corp). Such formulation was treated with the cat fecal matter and urine and evaluated via GC-MS analysis, as described in Example 1, and the results for headspace composition are given in Table 10.

TABLE 10
Headspace Composition Of Litter Formulation B4 (5 Wt %
Moderately Polar Porapak R Beads With Specific Surface
Area = 800-1000 M2/G Added To Clay Litter B) Assessed
With GC-MS Analytical Tool Coupled SPME Fiber Sampling

| RT | Library/ID | Qual | Area |
|---|---|---|---|
| 3.0657 | Hexane, 2,4-dimethyl- | 94 | 3074078 |
| 3.3741 | Benzene, methyl- (CAS) | 90 | 8460370 |
| 4.6758 | Cyclotrisiloxane, hexamethyl- | 90 | 15776201 |
| 5.0483 | Benzene, ethyl- (CAS | 81 | 2549859 |
| 7.0266 | Oxime-, methoxy-phenyl- | 83 | 44781565 |
| 7.3134 | Benzene, 1-ethyl-2-methyl- | 94 | 5918360 |
| 7.3563 | Benzene, 1-ethyl-2-methyl- | 94 | 4994472 |
| 8.4567 | Benzene, (2-methylpropyl)- | 91 | 8794624 |
| 8.521 | Benzene, (1-methylpropyl)- | 91 | 16444075 |
| 8.8678 | Cyclotetrasiloxane, octamethyl- (CAS) | 91 | 12738886 |
| 9.6086 | Benzene, 1,2-diethyl- | 96 | 1.46E+09 |
| 9.7456 | Benzene, 1,4-diethyl- (CAS) | 97 | 8.41E+08 |
| 9.8227 | Benzene, 1,2-diethyl- | 97 | 2.1E+08 |
| 9.9255 | Benzene, 1,4-diethyl- | 97 | 12568139 |
| 10.2766 | 1-Phenyl-1-butene | 95 | 5161585 |
| 10.4564 | Benzene, 1-ethenyl-3-ethyl-, mixt. | 90 | 2373768 |
| | with 1-ethenyl-4-ethylbenzene (CAS) | | |
| 11.7367 | Sulfamoxole | 87 | 4346752 |
| 11.9894 | Naphthalene, 1,2,3,4-tetrahydro- | 94 | 5661523 |
| 12.7473 | Benzene, 1,3,5-triethyl- | 87 | 7314627 |
| 12.8072 | Cyclopentasiloxane, decamethyl- | 91 | 7890021 |
| 12.9742 | Ethanone, 1-(4-ethylphenyl)- (CAS) | 94 | 6080937 |
| 14.109 | 1-Methyl-2-cyano-3-ethyl-2-piperideine | 80 | 6196346 |
| 14.383 | Ethanone, 1-(4-ethylphenyl)- (CAS) | 97 | 88225469 |
| 14.7983 | Ethanone, 1-(4-ethylphenyl)- (CAS) | 97 | 36516639 |
| 27.9568 | Sulfur, mol. (S8) (CAS) | 97 | 13228132 |

Referring to the Table 10, the data shows that the more polar beads of otherwise similar physical characteristics failed to absorb/adsorb as much of the malodor compounds found in the control sample B (Table 3) when compared to the more hydrophobic beads in Examples 1 to 4, 6, and 7. Therefore, these beads will not control malodors in a litter box.

Similar results are obtained with lower concentration of porous beads (2-3 wt % beads) added to clay litter A or clay litter B. Also, other porous materials, given in the Table 11, were tested and showed satisfactory malodor-controlling capability, when used in the tested conventional clay litters A and B.

TABLE 11
List Of Porous Materials Tested For Their Capability
To Adsorb/Absorb The Malodors In A Litter Box

| | | Specific | Avg. pore |
|---|---|---|---|
| Porous | | surface | diameter, |
| material | Composition | area, m²/g | nm |
| Purolite | Acid-treated cross-linked | 800-1000 | 85-95 |
| MN500 | polystyrene | | |
| Purolite | Base-treated cross-linked | 800-1000 | 85-95 |
| MN400 | polystyrene | | |
| Purolite | Not-functionalized hydrophobic | 800-1000 | 30-40 |
| MN250 | cross-linked polystyrene | | |
| Purolite | Not-functionalized hydrophobic | 800-1000 | 85-95 |
| MN200 | cross-linked polystyrene | | |
| Amberlite | Polystyrene divinylbenzene | 300 | 9-90 |
| XAD-2 | copolymer | | |
| Amberlite | Polystyrene | 784 | 50 |
| XAD-4 | | | |
| Amberlite | Cross-linked acrylic ester | 450 | 90 |
| XAD-7 | resin | | |
| Porapak-R | N-vinylpyrollidone | 550 | — |
| Porapak-T | Ethylene glycol | 300-450 | — |
| | dimethylacrylate | | |
| Porapak-Q | Ethylvinylbenzene- | 600 | 7.5-50 |
| | divinylbenzene | | |
| Porapak-P | Styrene-ethylvinylbenzene- | 50-100 | — |
| | divinylbenzene | | |
| Tenax TA | 2,6-diphenyleneoxide | 35 | 200 |
| Tenax GR | 2,6-diphenyleneoxide:graphite | 22 | — |
| | 70:30 | | |

In the specification, there have been disclosed typical preferred embodiments of the invention and, although specific terms are employed, they are used in a generic and descriptive sense only and not for purposes of limitation. Obviously many modifications and variations of the invention are possible in light of the above teachings. It is therefore to be understood that the invention may be practiced otherwise than as specifically described.

Unless defined otherwise, all technical and scientific terms and any acronyms used herein have the same meanings as commonly understood by one of ordinary skill in the art in the field of this invention. Although any compositions, methods, and means for communicating information similar or equivalent to those described herein can be used to practice this invention, the preferred compositions, methods, and means for communicating information are described herein.

All references cited above are incorporated herein by reference to the extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

What is claimed is:

1. A malodor control composition comprising:
a porous material comprising (1) a porous structure having surface area of from about 50 to about 1500 m³/g, (2) a pore diameter of from about 1 to about 200 nm, (3) a pore volume of from about 0.3 to about 1.5 cm³/g, and (4) a pronounced hydrophobicity with a surface contact angle greater than 100 degrees.

2. The malador control composition of claim 1 further comprising one or more sorbent materials.

3. The malodor control composition of claim 1 wherein the sorbent material is selected from the group consisting of activated carbon, baking soda, and combinations thereof.

4. An animal litter comprising:
one or more animal litters; and
one or more porous materials comprising (1) a porous structure having a surface area of from about 50 to about 1500 m²/g, (2) a pore diameter of from about 1 to about 200 nm, (3) a pore volume of from about 0.3 to about 1.5 cm³/g, and (4) a pronounced hydrophobicity with a surface contact angle greater than 100 degrees

5. The animal litter of claim 4 wherein the animal litter is selected from the group consisting of clays, woods, papers, grains, corncobs, seeds, and combinations thereof.

6. The animal litter of claim 4 wherein at least one of the animal litter and the porous material further comprises activated carbon.

7. The animal litter of claim 6 wherein the activated carbon comprises from about 0.01% to about 6% by weight of the animal litter and the porous material.

8. The animal litter of claim 4 wherein at least one of the animal litter and the porous material further comprises baking soda.

9. The animal litter of claim 8 wherein the baking soda comprises from about 0.01% to about 6% by weight of the animal litter and the porous material.

10. The animal litter of claim 4 wherein at least one of the animal litter and the porous material further comprises a partial or complete coating of one or more swelling clays.

11. The animal litter of claim 10 wherein the swelling clay is bentonite.

12. The animal litter of claim 10 wherein the swelling clay comprises from about 5 to about 40% by weight of the animal litter and the porous material.

13. The litter of claim 4 comprising an about 5 to about 95% of the animal litter and from 95% to about 5% of the porous material.

14. The animal litter of claim 4 wherein the animal litter is selected from the group consisting of swelling clay, non-swelling day, silica gel, and combinations thereof.

15. A method for making an animal litter comprising:
combining one or more an animal litters with one or more porous materials comprising (1) a porous structure having a surface area of from about 50 to about 1500 m²/g, (2) a pore diameter of from about 1 to about 200 nm, (3) a pore volume of from about 0.3 to about 1.5 cm³/g, and (4) a pronounced hydrophobicity with a surface contact angle greater than 100 degrees.

16. An animal litter box comprising:
a device suitable far containing animal litter and suitable for use by an animal when excreting animal waste; and
an animal litter comprising one or more animal litters and one or more porous materials comprising (1) a porous structure having a surface area of from about 50 to about 1500 m²/g, (2) a pore diameter of from about 1 to about 200 nm, (3) a pore volume of from about 0.3 to about 1.5 and (4) a pronounced hydrophobicity with a surface contact angle greater than 100 degrees.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. A method for controlling malodors comprising exposing a malodorous material to at least one malodor control composition of claim 1.

36. A method for controlling malodors from animal waste comprising exposing the animal waste to at least one malodor control composition of claim 1.

37. A method for controlling malodors from animal waste comprising exposing the animal waste to at least one animal litter of claim 4.
